(19) United States
(12) Patent Application Publication
(10) Pub. No.: US 2013/0331720 A1
(43) Pub. Date: Dec. 12, 2013

(54) ELECTROCARDIOGRAPH SYSTEM

(71) Applicants: Michael D. Battaglia, Philadelphia, PA (US); Sean M. Murphy, Oconomowoc, WI (US)

(72) Inventors: Michael D. Battaglia, Philadelphia, PA (US); Sean M. Murphy, Oconomowoc, WI (US)

(73) Assignee: TRIOMI MEDICAL INNOVATIONS, LLC, Miami, FL (US)

(21) Appl. No.: 13/493,968

(22) Filed: Jun. 11, 2012

(51) Int. Cl. A61B 5/044

(52) U.S. Cl. 600/523

(57) ABSTRACT

The present invention relates to an electrocardiograph (EKG) system and methods of using the same. The system includes a plurality of electrodes, an acquisition device and a display. The acquisition device includes a processor that is configured to process signals generated from the electrodes without receiving processing instructions from the display and/or the display includes a processor that is configured to check signals received from the acquisition device for errors prior to displaying on a display screen an electrocardiograph trace generated from signals received from the acquisition device

### BACKGROUND

1. Technical Field

The present invention relates to electrocardiograph (EKG) systems as well as to the methods of using the same.

2. Background of the Invention

Various EKG systems have been described in the prior art. For example, U.S. Pat. No. 7,236,818 describes a portable EKG system that includes a plurality of electrodes, an acquisition device and a display. As described in the '818 patent, the display and the acquisition device each include a processor, and the display controls the processing of data within the acquisition device. For example, in response to user inputs to a graphical user interface (GUI) located on the display, the display commands the acquisition device to collect data from the electrodes and uploads the data to the display.

Similarly, U.S. Pat. No. 8,082,027 describes a portable EKG system that includes a plurality of electrodes, an acquisition device, a display (referred to as a host device) and a USB cable that connects the acquisition device to the display. The EKG system of the '027 patent is described as being a multiplatform system, meaning that the acquisition device may work with displays having different operating systems. Like the '818 patent, the display and the acquisition device each include a processor and the display controls the processing of data within the acquisition device. For example, as described in the '027 patent, the acquisition device receives commands from a user operating the display and the commands initiate collecting EKG data from the patient.

However, there are several disadvantages with the EKG systems of the '818 and '027 patents. Indeed, by having the display control the acquisition device, the system has several points of failure. For example, the display may fail and send errant signals to the acquisition device. Further, the system is vulnerable to failures in communications from the display to the acquisition device. Given that EKG's are typically used in conjunction with very ill and/or high risk patients, a system that has multiple points of failure can be especially problematic.

Therefore, there is a need for new EKG systems that have improved protection against system failures.

### BRIEF SUMMARY

In certain aspects, the present invention provides an EKG system that includes a plurality of electrodes, an acquisition device and a display. The acquisition device is in communication with the electrodes when the system is in use and includes an acquisition device signal processor. The display is preferably a computer and includes a display screen configured to display an electrocardiograph trace generated from signals received from the acquisition device, a memory, a processor, and an operating system. In some embodiments, the acquisition device signal processor is configured to process signals received from the plurality of electrodes without receiving processing instructions from the display. In addition to or in leiu of having the acquisition device signal processor configured to process signals received f′rom the plurality of electrodes without receiving processing instructions from the display, in some embodiments, the display is configured to check for errors in signals received from the acquisition device prior to displaying on the display screen an electrocardiograph trace generated from signals received from the acquisition device. Optionally, the display processor is further configured to correct errors in signals received from the acquisition device prior to displaying on the display screen an electrocardiograph trace generated from signals received from the acquisition device.

In some embodiments the display is in direct communication with the acquisition device (e.g., directly cohnected to the acquisition device via a cable or a wireless connection). In some embodiments, the acquisition device further includes a communications processor configured to establish communications between the display and the acquisition device. In some embodiments, the acquisition device is configured to filter signals in the acquisition device without receiving filtering instructions from the display.

In some embodiments, the electrocardiograph system is portable and the display and the acquisition device are each capable of being carried by a human adult in one hand.

Without being bound to any particular theory, it is believed that the systems of the present disclosure improve data integrity by reducing the points of failure of the system. Thus, in certain embodiments the present disclosure is directed to a method of improving data integrity that includes:
a) providing the electrocardiograph system;
b) placing the plurality of electrodes on the body of an animal;
c) transmitting signals from the plurality of electrodes to the acquisition device signal processor;
d) transmitting signals from the acquisition device to the display; and
e) displaying on the display screen an electrocardiograph trace generated from signals received from the acquisition device.

In some embodiments, the method further includes processing signals received from the plurality of electrodes in the acquisition device signal processor without receiving processing instructions from the display. In addition to or in lieu of processing signals received from the plurality of electrodes in the acquisition device signal processor without receiving processing instructions from the display, in some embodiments, the method further includes using the display processor to check signals received from the acquisition device for errors prior to displaying on the display screen an electrocardiograph trace generated from signals received from the acquisition device. Optionally, the method further includes using the display processor to correct errors in signals received from the acquisition device prior to displaying on the display screen an electrocardiograph trace generated from signals received from the acquisition device. Optionally, the method further includes sending signals from the display to the acquisition device to establish device recognition between the display and the acquisition device and thereafter ceasing all communications from the display to the acquisition device.

### BRIEF DESCRIPTION OF THE DRAWINGS

FIG. 1 illustrates a top view of one embodiment of a portable, electrocardiograph system.

FIG. 2 illustrates a schematic view of an embodiment of an electrocardiograph system.

FIG. 3 illustrates a schematic view of an embodiment of an electrocardiograph system.

FIG. 4 illustrates a schematic view of an embodiment of an electrocardiograph system.

FIG. 5 illustrates a schematic view of an embodiment of an electrocardiograph system.

FIG. 6 illustrates a schematic view of an embodiment of an electrocardiograph system.

FIG. 7 illustrates a schematic view of an embodiment of an electrocardiograph system.

### DETAILED DESCRIPTION

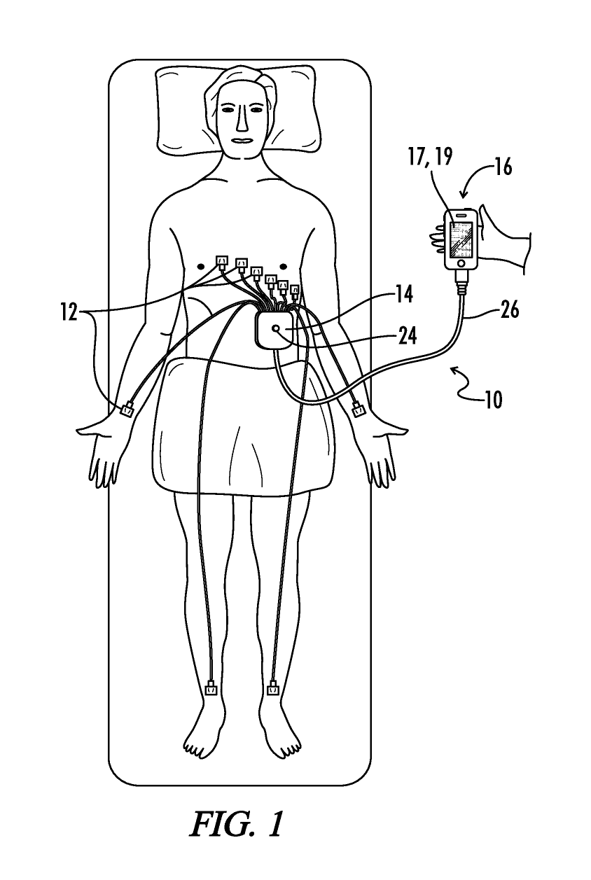

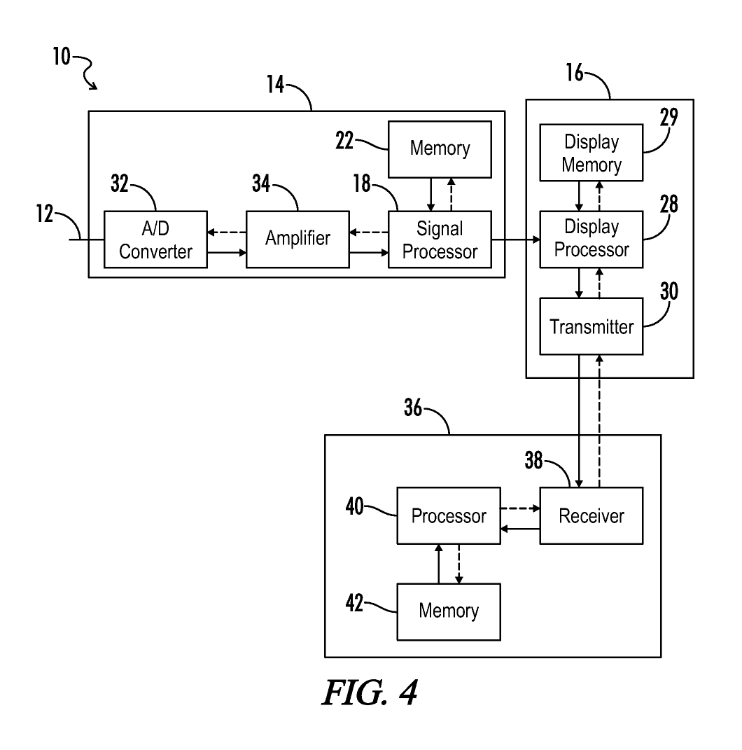

Referring now to the drawings, FIGS. 1-7 illustrate several embodiments of an EKG system generally designated by the numeral 10. In the drawings, not all reference numbers are included in each drawing for the sake of clarity.

Referring further to FIG. 1, the EKG system 10 includes a plurality of electrodes 12, an acquisition device 14 and a display 16. Electrodes for use in connection with the present invention include those now known and later developed for use in conjunction with EKG systems. Optionally, the system 10 includes at least ten electrodes, as EKG systems including ten electrodes are used to generate 12-lead and 15-lead electrocardiograms. However, the system 10 may include more or fewer electrodes, depending on the needs of the user (e.g., the paramedic, doctor or other caregiver) operating the system 10.

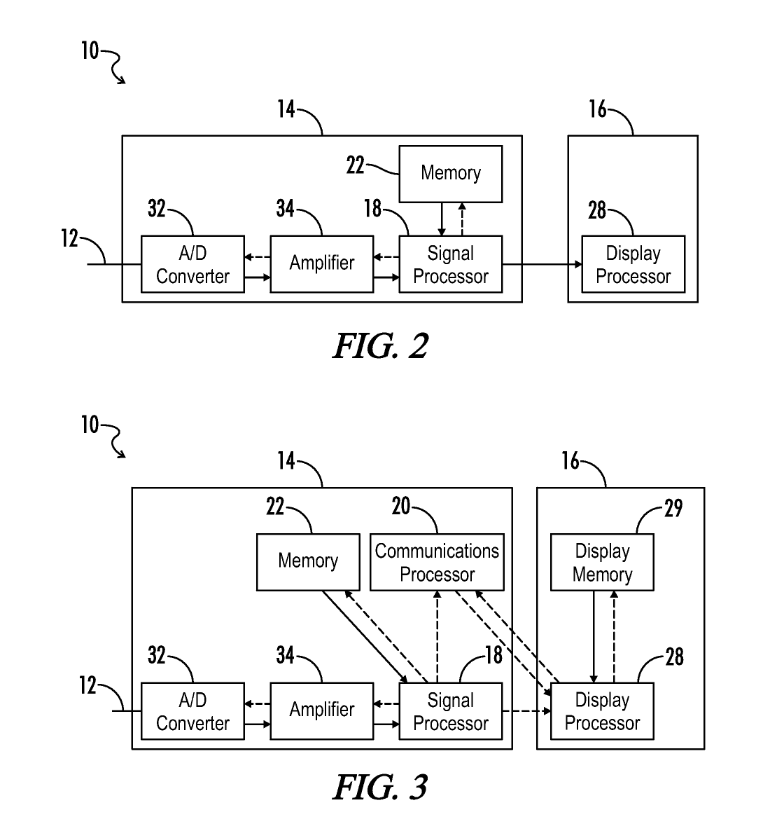

The system 10 further includes an acquisition device 14. The acquisition device 14 includes an acquisition device signal processor 18 and an acquisition device memory 22. Preferably, the acquisition device 14 further includes an analog-digital converter 32 and an amplifier 34. Diagrams illustrating the flow of signals in the acquisition device 12 are shown in FIGS. 2-7, where a solid arrow means that the flow of signals in a particular direction is mandatory for the particular embodiment illustrated and a dashed arrow means that the flow of signals in a particular direction is optional for the particular embodiment illustrated. For example, in the embodiments shown in FIGS. 2-7, analog signals received from the electrodes 12 are converted into digital signals by the analog-digital converter 32 and then amplified by the amplifier 34 prior to being processed by the acquisition device signal processor 18. However, it will be understood that the arrangements shown are merely exemplary and, for example, signals received from the electrodes 12 may be amplified by the amplifier 34 prior to being converted into digital signals by the analog-digital converter 32.

The acquisition device signal processor 18 is a processor located in the acquisition device 14 that processes the signals received from the electrodes 12 pursuant to software instructions stored on the acquisition device memory 22. Preferably, as mentioned above, the signals received from the electrodes 12 are converted into digital signals by the analog-digital converter 32 prior to being processed by the acquisition device signal processor 18.

In some embodiments, the acquisition device memory 22 is a removable data storage medium (e.g., a flash card, memory stick, CD) so that a user may be able to easily update the software program. In other embodiments, the acquisition device memory 22 is a non-removable data storage medium (e.g., a hard drive). Optionally, the acquisition device memory 22 is configured to store processed signals generated by the acquisition device signal processor 18, as indicated by the dashed arrows in FIGS. 2-7. Alternatively, the acquisition device 12 may include a second memory (not shown) that is configured to store processed signals generated by the acquisition device signal processor 18.

Optionally, the acquisition device signal processor 18 controls the acquisition of signals from the electrodes and the flow of signals through the analog-digital converter 32 and amplifier 34, as indicated in the dashed arrows in FIGS. 2-7. Optionally, the acquisition device signal processor 18 is a digital signal processor (DSP), such as the TMS320C5515 digital signal processor produced by Texas Instruments, Dallas, Tex.

In use, the acquisition device 14 is coupled to the display 16 (e.g., via a wireless connection or a cable 26). Preferably, the display 16 is in “direct communication” with the acquisition device 14, meaning that signals are transmitted from the acquisition device 14 to the display 16 without passing through another device that further processes the signals. In other words, when the display 16 is in direct communication with the acquisition device 14, a signal transmitted from the acquisition device 14 is not processed by another device (i.e, a device other than the acquisition device 14 and the display 16) before the signal reaches the display 16. In some embodiments, the acquisition device 14 further includes a radio transmitter (not shown) (e.g., a radio receiver or transceiver) to wirelessly transmit signals from the acquisition device 14 to the display 16. Optionally, the acquisition device 14 may transmit signals to the display 16 via Bluetooth wireless communication.

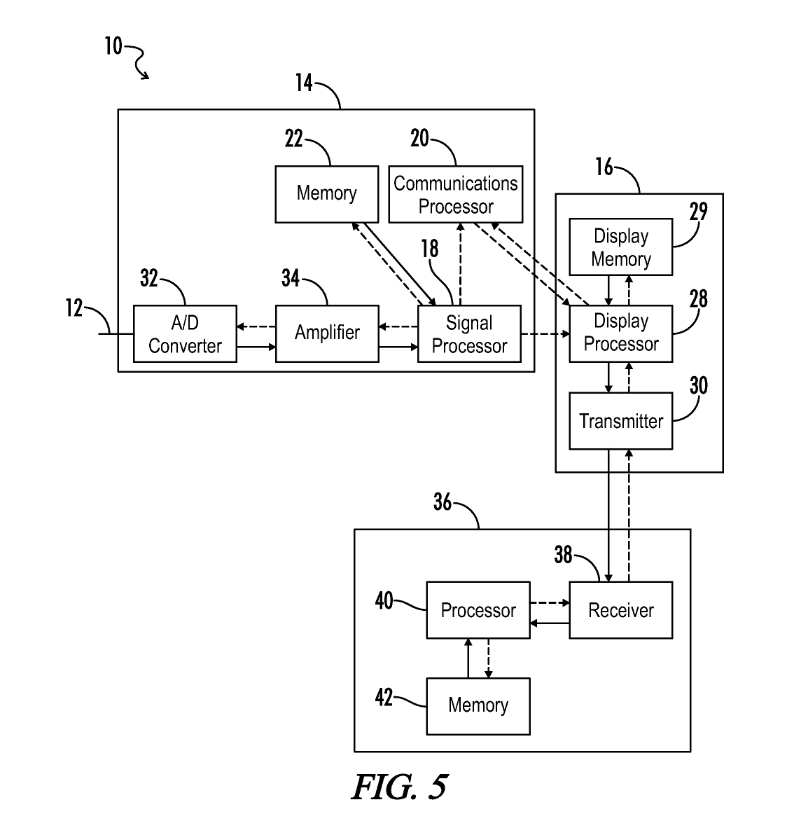

In some embodiments, as exemplified in FIGS. 3 and 5, the acquisition device 14 further includes a communications processor 20 that establishes device recognition and/or helps manage communications between the acquisition device 14 and the display 16. As indicated by the dashed arrows, in FIGS. 3 and 5, if a communications processor 20 is included, signals may or may not flow from the acquisition device signal processor 18 to the communication processor 20 prior to being transmitted to the display 16. Preferably, if a communications processor 20 is included, signals flow from the acquisition device signal processor 18 to the communication processor 20 prior to being transmitted to the display 16.

In an exemplary embodiment, the acquisition device signal processor 18 sends output data to a Universal Asynchronous Receiver/Transmitter (UART). In the exemplary embodiment, only the Transmitter portion of the UART is used; the Receiver portion is not used. The UART is coupled to a communications processor 20 that handles all communications between the display 16 and the acquisition device 14 that the communications protocol necessitates and thus keeps the processing by the acquisition device signal processor 18 and the processing by the display processor 28 completely separate. For example, the communications processor 20 may be a UART→USB converter IC or a UART→Bluetooth converter. In such embodiments, the communications processor 20 will handle the formatting of signals within the USB or Bluetooth standard and will handle any USB or Bluetooth-related backflow coming from the display 16 dealing with USB or Bluetooth enumeration without sending the backflow onto the acquisition device signal processor 18. It will be appreciated that the USB and Bluetooth protocols are merely exemplary and other protocols may be used in conjunction with the present invention.

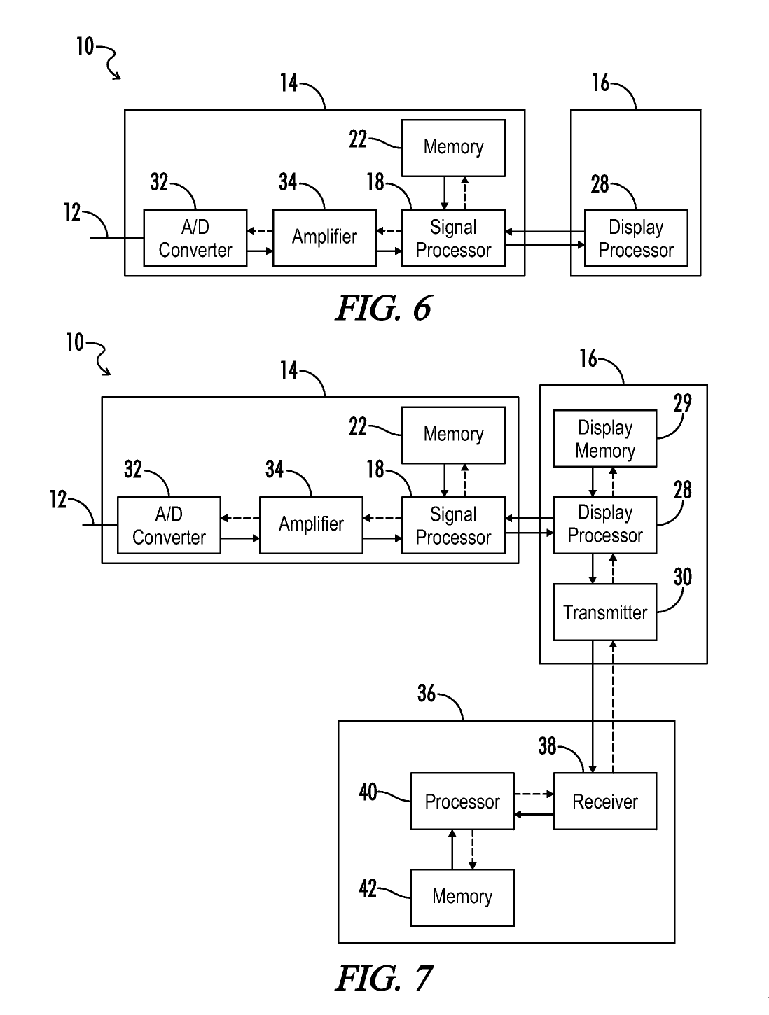

In another embodiment, exemplified in FIGS. 6 and 7, the system 10 does not include a communications processor 20 but the acquisition device signal processor 18 has the built-in ability to handle protocol (e.g., USB or Bluetooth)-related communications from the display 16 to the acquisition device 14, such as metadata communication protocols and formatting of signals within the USB or Bluetooth standard. If such a configuration is used, the display 16 preferably will send only protocol-related communications to the acquisition device 14, the display 16 will not instruct the acquisition device 14 to transmit EKG-related signals to the display 16 and the display 16 will not instruct the acquisition device signal processor 18 to alter how the acquisition device signal processor 18 is processing the electrode signals.

Optionally, the acquisition device 14 further includes a warning light or warning signal 24 to warn an operator of any abnormalities associated with signals generated from the electrodes 12. For example, in one embodiment, the warning light or signal 24 may simply inform an operator that one or more of the electrodes 12 is not connected to the proper location on a patient's body. Alternatively, the warning light or signal 24 may warn inform an operator that the acquisition device signal processor 18 has detected abnormalities concerning a patient's heart.

Preferably, the acquisition device 14 further includes a power source (not shown) and power switch (not shown) to power the acquisition device 14. Preferably, the power source is a battery.

The system 10 further includes a display 16. Preferably, the display 16 is a computer. As used herein, the term “computer” means a multi-purpose device that includes a processor, an operating system and a memory that has stored in the memory EKG-related and non-EKG-related software. By “multi-purpose” device, it is meant that the display computer 16 can be used for both EKG-related and non-EKG related purposes. The display computer 16 may be, for example, a desktop computer, a laptop computer or a mobile telephone.

The display 16 includes one or more display screens 17 configured to display an electrocardiograph trace generated from signals received from the acquisition device 14. As shown, in FIGS. 2-7, the acquisition device 14 and display 16 are discrete units and the components of each of the acquisition device 14 and display 16 are located in separate housings. Optionally, the acquisition device 14 and display 16 removably attach to each other. For example, in some embodiments, the acquisition device 14 may be a clocking station that can removably receive the display 16. Alternatively, the acquisition device 14 may snap on to the display 16.

The display 16 further includes a display processor 28 that handles signals received from the acquisition device 14. Preferably, the display processor 28 processes the digital signals pursuant to software instructions stored on the display memory 29. In some embodiments, the display memory 29 is a removable data storage medium (e.g., a flash card, memory stick, CD) so that a user may be able to easily update the software program. In other embodiments, the display memory 29 is a non-removable data storage medium (e.g., a hard drive). Optionally, the display memory 29 is configured to store signals received from the acquisition device 14. Alternatively, the display 16 may include a second memory (not shown) that is configured to store processed signals transmitted from the acquisition device 14. Optionally, prior to being stored on the display memory 29, the signals are processed in the display processor 28. In addition to the display screen 17, the display processor 28, and the display memory 29, the display 16 optionally includes a radio transmitter 30 (e.g., a radio receiver or transceiver) to transmit signals from the display 16 to another computer 36 (e.g., a mobile telephone, laptop computer or desktop computer), and optionally to receive signals from the other computer 36, to, for example, enable remote (i.e, off-site) monitoring of a patient. In such embodiments, the second computer 36 further includes a processor 40, a memory 42, and a radio receiver 38 (e.g., a radio receiver or transceiver). In some embodiments, the processor 40 simply runs a server (e.g., an HTTP server) that displays the information transmitted from the display 16. In other embodiments, the processor 40 further processes the signals received from the display 16. Optionally, the processor 40 processes the signals pursuant to software instructions stored on the memory 42. In some embodiments, the memory 42 is a removable data storage medium (e.g., a flash card, memory stick, CD) so that a user may be able to easily update the software program. In other embodiments, the memory 42 is a non-removable data storage medium (e.g., a hard drive). Optionally, the memory 42 is configured to store signals transmitted from the display 16. Alternatively, the second computer 36 may include a second memory (not shown) that is configured to store processed signals transmitted from the display 16. Optionally, prior to being stored on the computer memory 42, the digital signals are processed on the processor 40.

Optionally, the display 16 and the second computer 36 run on a Windows®, Macintosh®, iPhone®, Android®, and/or Linux® operating system.

Optionally, the display 16 includes a graphical user interface (GUI) 19. Preferably, the display screen 17 and GUI 19 are a single screen, as shown in FIG. 1.

Preferably, the display 16 is a mobile telephone (e.g., an iPhone®, Android®, etc.) that can be held in the palm of an operator's hand. Preferably, the display 16 is used in close proximity to the acquisition device 14. For example, preferably, the display 16 is used within about fifteen feet or less of the acquisition device 14 so that an operator can make an on-the-spot diagnosis of a patient.

As explained in the “Background”, having the display 16 control the acquisition device 14 can be problematic. Thus, in certain embodiments, the systems 10 of the present disclosure have limited (if any) communications from the display 16 to the acquisition device 14, particularly when the acquisition signal processor 18 is processing signals received from the electrodes 12. For example, preferably, the acquisition device processor 18 is configured to process signals received from the electrodes 12 pursuant to instructions stored on the acquisition device memory 22 without receiving any processing instructions from the display 16. Preferably, the acquisition device processor 18 is configured to process signals received from the electrodes 12 without receiving any input from the display 16. Preferably, the acquisition device 14 is configured to filter signals in the acquisition device 14 and does so without receiving any filtering instructions from the display 16. Optionally, in such embodiments, the acquisition device 14 further includes a communications processor 20 to manage communications from the acquisition device 14 to the display 16, as illustrated in FIGS. 3 and 5. However, preferably, in such embodiments, the communications processor 22 does not send any signals to the acquisition device signal processor 18. Alternatively, as explained previously and illustrated in FIGS. 6 and 7, the acquisition device signal processor 18 may receive only protocol-related communications from the display 16 and, in such embodiments, the display 16 will send only protocol-related communications to the acquisition device 14, the display 16 will not instruct the acquisition device 14 to transmit EKG-related signals to the display 16 and the display 16 will not instruct the acquisition device signal processor 18 to alter how the acquisition device signal processor 18 is processing the electrode signals. It is further preferred, in certain embodiments, that after sending signals from the display 16 to the acquisition device 14 to establish device recognition between the display 16 and the acquisition device 14 (e.g., a digital handshake), no further signals are sent from the display 16 to the acquisition device 14 while the acquisition device signal processor 18 is processing signals received from the electrodes 12.

Given that the acquisition device signal processor 18 operates independently from the display processor 28 in certain embodiments and the acquisition device signal processor 18 can't request a do-over for signals that have been dropped, optionally the system 10 includes methods to ensure the integrity of the signals being sent from the acquisition device 14 to the display 16. In some embodiments, such methods include using the display processor 28 to check signals received from the acquisition device 14 for errors prior to displaying on the display screen 17 an electrocardiograph trace generated from signals received from the acquisition device 14. Optionally, such methods further include using the display processor 28 to correct errors in signals received from the acquisition device 14 prior to displaying on the display screen 17 an electrocardiograph trace generated from signals received from the acquisition device 14.

For example, in some embodiments, the acquisition device 14 implements a checksum routine which gives each data packet a “fingerprint” and the display processor 28 checks the packet against the fingerprint. If the fingerprint doesn't match the packet, the display 16 knows something is wrong and doesn't display the packet, treating it as an error. In other embodiments, the display processor 28 implements forward error correction through an error-correcting code such as the Reed-Solomon code to dynamically reconstruct lost bits in dropped packets, similar to the way a CD player reconstructs lost samples if the CD is scratched. In further embodiments, the display processor 28 may attempt to interpolate the surrounding samples to guess at the nature of the dropped packets.

The functions performed by the acquisition signal processor 18 and the display processor 28 may vary depending on, for example, the requirements of the user. For example, in certain embodiments, the acquisition signal processor 28 may control the collection of signals from the electrodes 12, filter the signals, and perform all or substantially all calculations needed to construct an electrocardiograph trace and, optionally, to calculate the patient's heart rate. The acquisition signal processor 18 may also execute software that allows the acquisition signal processor 18 to diagnose the patient's heart condition based on the electrode signals. In such embodiments, the display 16 may do little more than execute a software viewing program that displays an electrocardiograph trace and, optionally, a heart rate and a warning, based on the signals transmitted from the acquisition device 14. In other embodiments, the acquisition signal processor 28 may still control the collection of signals from the electrodes 12 and may still perform some of the calculations needed to construct an electrocardiograph trace and, optionally, to calculate the patient's heart rate, however, the display 16 may handle signal filtering, a significant portion of the calculations needed to construct an electrocardiograph trace and, optionally, to calculate the patient's heart rate. The display processor 28 may further execute software that allows the display processor 28 to diagnose the patient's heart condition based on the electrode signals. In either embodiment, preferably, the display 16 allows a user to send the electrocardiograph signals from the display 16 to a second computer 36.

In some embodiments, the entire system 10 is portable, as shown in FIG. 1. As used herein, the term “portable” means that the entire system 10 is capable of being carried by a human adult with two hands. In such embodiments, preferably the acquisition device 14 and display 16 are handheld. The term “handheld”, as used herein, means the individual component (e.g., the acquisition device 14 or the display 16) is capable of being carried by a human adult with one hand. Optionally, the weight of the display 16 is about one pound or less.

Without being bound to any particular theory, it is believed the systems 10 of the present disclosure increase data integrity by reducing the points of failure, as compared to certain prior art EKG systems. Thus, in certain embodiments, the present disclosure is directed to using the system 10 to improve data integrity. In certain embodiments, the method includes:
a) placing the plurality of electrodes 12 on the body of an animal;
b) transmitting signals from the plurality of electrodes 12 to the acquisition device signal processor 18;
c) transmitting signals from the acquisition device 12 to the display 16; and
d) displaying on the display screen 17 an electrocardiograph trace generated from signals received from the acquisition device 14.

In some embodiments, the method further includes: processing signals received from the plurality of electrodes 12 in the acquisition device signal processor 18 without receiving processing instructions from the display 16. In addition to or in lieu of processing signals received from the plurality of electrodes 12 in the acquisition device signal processor 18 without receiving processing instructions from the display 16, in some embodiments, the method further includes: using the display processor 28 to check signals received from the acquisition device 14 for errors prior to displaying on the display screen 17 an electrocardiograph trace generated from signals received from the acquisition device 14. Optionally, the method further includes using the display processor 28 to correct errors in signals received from the acquisition device 14 prior to displaying on the display screen 17 an electrocardiograph trace generated from signals received from the acquisition device 14. Optionally, the method further includes sending signals from the display 16 to the acquisition device 14 to establish device recognition between the display 16 and the acquisition device 14 and thereafter ceasing all communications from the display 16 to the acquisition device 14. Optionally, the method further includes using software stored in the display 16 to analyze signals received from the acquisition device 14.

Optionally, the system 10 is used for continuous monitoring of persons, e.g., patients having a risk or known cardiac problems and/or persons engaged in high risk professions, such as fork lift operators or pilots.

Optionally, the display 16 is configured to display the complete 12/15-lead electrocardiograph trace with hashmarks on the display screen 17 as well as the patient's heart rate and further allows a user to choose and/or zoom in on individual leads. Optionally, the display screen 17 is configured to display all 12 or 15 leads and tapping on a particular lead zooms in on that lead and then a user may swipe the screen 17 to display the next lead.

Optionally, the electrocardiograph trace displayed on the display screen 17 is able to be saved in memory located in the display 16. Optionally, the electrocardiograph trace and/or other signals received from the acquisition device 14 is sent to a second computer 36 with or without saving the electrocardiograph trace and/or other signals in memory located in the display 16. Optionally, the display 16 is configured to detect printers in range of the display 16 and print to such printers. Optionally, the display 16 includes software for automatically interpreting the signals received from the acquisition device 14. Optionally, in such embodiments, the display 16 may issue a warning based on an animal's heart condition.

Optionally, the system 10 includes a physical diagram and/or a diagram that is displayed on the display screen 17 that instructs a user where to place the electrodes 12 on the body of an animal.

Having now described the invention in accordance with the requirements of the patent statutes, those skilled in the art will understand how to make changes and modifications to the disclosed embodiments to meet their specific requirements or conditions. Changes and modifications may be made without departing from the scope and spirit of the invention, as defined and limited solely by the following claims.

1. An electrocardiograph system comprising:
a) a plurality of electrodes;
b) an acquisition device comprising an acquisition device signal processor configured to process signals received from the plurality of electrodes; and
c) a display computer in direct communication with the acquisition device, the display computer comprising a display screen configured to display an electrocardiograph trace generated from signals received from the acquisition device, a memory, a processor, and an operating system,
wherein the acquisition device signal processor is configured to process signals received from the plurality of electrodes and further wherein the acquisition device signal processor is configured to perform all acquisition device signal processor signal processing without receiving any processing instructions from the display computer.

2. The electrocardiograph system of claim 1 further comprising a cable for connecting the acquisition device to the display computer.

3. The electrocardiograph system of claim 1, wherein the acquisition device further comprises a communications processor configured to establish communications between the display computer and the acquisition device without the communications processor sending any signals to the acquisition device signal processor.

4. The electrocardiograph system of claim 1, wherein the electrocardiograph system is portable and the display computer and the acquisition device are each capable of being carried by a human adult in one hand.

5. The electrocardiograph system of claim 1, wherein the display computer is a mobile telephone that further comprises a graphical user interface.

6. The electrocardiograph system of claim 1, wherein the acquisition device is configured to filter signals in the acquisition device without receiving filtering instructions from the display computer.

7. The electrocardiograph system of claim 1, wherein the display processor is configured to check signals received from the acquisition device for errors prior to displaying on the display screen an electrocardiograph trace generated from signals received from the acquisition device.

8. The electrocardiograph system of claim 1, wherein the display processor is configured to correct errors in signals received from the acquisition device prior to displaying on the display screen an electrocardiograph trace generated from signals received from the acquisition device.

9. The electrocardiograph system of claim 1, wherein the weight of the display computer is less than about one pound.

10. The electrocardiograph system of claim 1, wherein the display computer is configured to transmit signals from the display computer to another computer.

11. A method of improving data integrity, the method comprising:
a) providing the electrocardiograph system of claim 1;
b) placing the plurality of electrodes on the body of an animal;
c) transmitting signals from the plurality of electrodes to the acquisition device signal processor;
d) processing signals received from the plurality of electrodes in the acquisition device signal processor without receiving processing instructions from the display computer;
e) transmitting signals from the acquisition device to the display computer; and
f) displaying on the display screen an electrocardiograph trace generated from signals received from the acquisition device,
wherein all acquisition device signal processor signal processing is performed without receiving any processing instructions from the display computer.

12. The method of claim 11, wherein the method further comprises sending signals from the display computer to the acquisition device to establish device recognition between the display computer and the acquisition device and then ceasing all communications from the display computer to the acquisition device while the acquisition device signal processor is processing signals received from the electrodes.

13. The method of claim 11, further comprising using software stored in the display computer to analyze signals received from the acquisition device.

14. A portable electrocardiograph system comprising:
a) a plurality of electrodes;
b) an acquisition device comprising an acquisition device signal processor configured to process signals received from the plurality of electrodes; and
c) a display computer in direct communication with the acquisition device, the display computer comprising a display screen configured to display an electrocardiograph trace generated from signals received from the acquisition device, a memory, a processor, and an operating system,
wherein the display processor is configured to check signals received from the handheld acquisition device for errors prior to displaying on the display screen an electrocardiograph trace generated from signals received from the acquisition device.

15. The electrocardiograph system of claim 14, wherein the display processor is configured to correct errors in signals received from the acquisition device prior to displaying on the display screen an electrocardiograph trace generated from signals received from the acquisition device.

16. The electrocardiograph system of claim 14, wherein the acquisition device processor is configured to process signals received from the plurality of electrodes without receiving processing instructions from the display computer.

17. The electrocardiograph system of claim 14 further comprising a cable for connecting the acquisition device to the display computer.

18. The electrocardiograph system of claim 14, wherein the acquisition device further comprises a communications processor configured to establish communications between the display computer and the acquisition device.

19. The electrocardiograph system of claim 14, wherein the electrocardiograph system is portable and the display computer and the acquisition device are each capable of being carried by a human adult in one hand.

20. The electrocardiograph system of claim 14, wherein the display computer is a mobile telephone that further comprises a graphical user interface.

21. The electrocardiograph system of claim 14, wherein the acquisition device is configured to filter signals in the acquisition device without receiving filtering instructions from the display computer.

22. The electrocardiograph system of claim 14, wherein the weight of the display computer is less than about one pound.

23. The electrocardiograph system of claim 14, wherein the display computer is further configured to transmit from the display computer to another computer.

24. A method of improving data integrity comprising:
a) providing the electrocardiograph system of claim 14;
b) placing the plurality of electrodes on the body of an animal;
c) transmitting signals from the plurality of electrodes to the acquisition device signal processor;
d) processing signals received from the plurality of electrodes in the acquisition device signal processor;
e) transmitting signals from the acquisition device to the display computer;
f) checking signals received from the acquisition device for errors using the display processor; and
g) displaying on the display screen an electrocardiograph trace generated from signals received from the acquisition device.

25. A portable electrocardiograph system comprising:
a) a plurality of electrodes;
b) a handheld acquisition device comprising an acquisition device signal processor configured to process signals received from the plurality of electrodes; and
c) a handheld display, the handheld display comprising a display screen configured to display an electrocardiograph trace generated from signals received from the acquisition device, a memory, a processor, and an operating system, wherein the display processor is configured to check signals received from the acquisition device for errors prior to displaying on the display screen an electrocardiograph trace generated from signals received from the acquisition device.

26. The portable electrocardiograph system of claim 25, wherein the acquisition device processor is configured to process signals received from the plurality of electrodes without receiving processing instructions from the display.

27. The portable electrocardiograph system of claim 25, wherein the display is a computer.

28. The portable electrocardiograph system of claim 25, wherein the display processor is configured to correct errors in signals received from the acquisition device prior to displaying on the display screen an electrocardiograph trace generated from signals received from the acquisition device.

29. A method of improving data integrity comprising:
a) providing the portable electrocardiograph system of claim 25;
b) placing the plurality of electrodes on the body of an animal;
c) transmitting signals from the plurality of electrodes to the acquisition device signal processor;
d) processing signals received from the plurality of electrodes in the acquisition device signal processor;
e) transmitting signals from the acquisition device to the display; and
f) checking signals received from the acquisition device for errors using the display processor.

30. The method of claim 11, wherein the method further comprises the step of checking signals received from the acquisition device for errors using the display processor.

31. The method of claim 30, wherein the display processor uses a checksum to detect errors in signals received from the acquisition device.
